(19) United States
(12) Patent Application Publication
(10) Pub. No.: US 2018/0055871 A1
(43) Pub. Date: Mar. 1, 2018

(54) Nutritional composition or pharmaceutical and dietary supplement containing said nutritional or pharmaceutical composition

(71) Applicant: Pharmunion, LLC, Wilmington, DE (US)

(72) Inventor: Daniele Scatolero, Piasco (IT)

(73) Assignee: Pharmunion, LLC, Wilmington, DE (US)

(21) Appl. No.: 15/685,441

(22) Filed: Aug. 24, 2017

(30) Foreign Application Priority Data
Aug. 31, 2016 (IT) 102016000088648

(51) Int. Cl. A61K 31/728; A61K 31/734; A23L 33/00

(52) U.S. Cl. CPC A61K 31/728; A61K 31/734; A23L 33/00; A23V 2250/5026; A23V 2002/00; A23V 2250/308; A61K 2300/00

(57) ABSTRACT

The present invention relates to a nutritional or pharmaceutical composition for the treatment or prevention of gastroesophageal reflux disease comprising at least one salt of alginic acid and a salt of hyaluronic acid. The nutritional or pharmaceutical composition according to the invention has the advantage of being able to be used for the treatment or prevention of gastroesophageal reflux disease by acting, through the formation of a spongy gel, as a protective barrier that mechanically protects the walls of the gastroesophageal mucosa from possible erosion by acid in the contents of the stomach. According to the invention, a dietary supplement that comprises said nutritional or pharmaceutical composition for the treatment or prevention of gastroesophageal reflux disease is also provided.

### CROSS-REFERENCE TO RELATED APPLICATIONS

This application is a nonprovisional utility application of the patent application, serial number 102016000088648, filed in the Italian Office Patents and Trademarks on Aug. 31, 2016 and claims the priority thereof and is expressly incorporated herein by reference in its entirety.

### TECHNICAL FIELD

The present invention relates to a nutritional or pharmaceutical composition for the treatment or prevention of gastroesophageal reflux disease; the present invention relates to a nutritional or pharmaceutical composition for the treatment or prevention of gastroesophageal reflux disease that allows you to mechanically protect the esophagus from the stomach acid reflux (gastroesophageal reflux).

The present invention further relates to a dietary supplement containing the above composition, wherein said integrator—allowing to mechanically protect the esophagus from the stomach acid reflux—can be used for medical purposes.

### BACKGROUND

Gastroesophageal reflux is substantially a phenomenon characterized by the temporary rise of acid content of the stomach into the esophagus, without necessarily causing regurgitation or vomiting.

The physiological gastroesophageal reflux is common in children, especially in the first months of life, but it also presents in healthy adults. Usually it lasts less than 3 minutes and prevails in the postprandial periods. When it occurs, the contents of the stomach acid can be felt near the rear of the mouth, causing a temporary burning sensation on the palate, throat or heartburn.

However, when the reflux is more frequent, or when the contents of the stomach is too acidic, the reflux becomes a chronic disorder. One speaks in this case of gastroesophageal reflux disease, a pathological condition characterized by a combination of typical symptoms and/or endoscopic evidence of injury to the mucosa of the esophageal wall, caused by the same reflux.

During eating, the esophagus, aided by gravity and a series of peristaltic muscle movements, advances the swallowed food (bolus) down. The passage of the food bolus in the stomach is regulated by the lower esophageal sphincter, a muscular valve that opens and allows the passage of the bolus of food from the esophagus to the stomach. It is precisely this sphincter, which upon closing, prevents the ascent upward of the acid contents present in the stomach.

Gastroesophageal reflux occurs when the lower esophageal sphincter opens at a time that is not appropriate, allowing the passage upward of acidic stomach contents into the esophagus. By virtue of its acidity, such content irritates the mucosa of the esophageal wall, triggering the aforementioned typical symptoms.

The opening of the esophageal sphincter can occur at inappropriate times, due to insufficient strength of the latter with respect to an increase in abdominal pressure, or to a progressive weakening of the sphincter closing pressure.

The objective of gastroesophageal reflux disease therapy is symptom control. This type of therapy does not treat the etiology of the disease (malfunction of the lower esophageal sphincter), but the courses of action largely follow according to two principle lines: the first principle involves mechanically interposing a substance between the contents of the stomach acid and esophageal walls in order to limit the time of exposure of the esophageal mucosa of the wall to the acid contents of the stomach; the second principle attempts to counteract the acidity by making the contents of the stomach acid the least possible detrimental.

With regard to the first principle of the above-mentioned course of action, in the prior art, it is known that there are nutritional or pharmaceutical compositions capable of mechanically interposing between the acid contents of the stomach and esophageal walls. By way of example, there are nutritional or pharmaceutical compositions based on the so-called alginates, namely salts of alginic acid, and in particular, of sodium alginate.

However, it should be considered that when the sodium alginate arrives in the stomach, and comes into contact with its acid content, it forms a water-insoluble polymer which tends to mix with the food and precipitate within the acidic stomach contents, passing in this way inside the intestine, rather than towards the walls of the esophagus.

In order for the action of the sodium alginate interposed mechanically between the acid contents of the stomach and esophageal walls to be effective, the alginate polymer must be able to place on top of the stomach contents, and not inside it, so as to form a sort of “floating barrier”.

In order to facilitate the flotation of the alginate polymer, for example, the technique of administering sodium alginate in a combined formulation with sodium or potassium bicarbonate is known.

The bicarbonate in contact with hydrochloric acid, present in the acidic stomach contents generates free gaseous carbon dioxide which pushes the alginate polymer upward. However, the use of a gas (carbon dioxide) is not optimal and may not cause the newly formed polymer to float above the acid content. The concentration of bicarbonate in fact influences the gas formation rate, which constitutes a critical factor. Too fast or too slow development of carbon dioxide facilitates the dispersion of the gas instead of its interaction with the alginate polymer.

While these methods may be suitable for the particular purpose employed, or for general use, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

The purpose of the present invention is to provide a nutritional or a pharmaceutical composition and a corresponding dietary supplement containing said composition that overcome the drawbacks of prior art and making it possible to form in a rapid and effective manner, a mechanical barrier between the contents of the stomach acid and esophageal walls, so as to control the symptoms caused by gastroesophageal reflux disease.

This and other objects are achieved by means of a nutritional or pharmaceutical composition and a dietary supplement as claimed in the appended claims.

The present disclosure addresses at least one of the foregoing disadvantages of the prior art. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### DETAILED DESCRIPTION OF THE PREFERRED EMBODIMENTS

According to the invention there is provided a nutritional or pharmaceutical composition for the treatment or prevention of gastroesophageal reflux disease which comprises at least one salt of alginic acid and that, advantageously, further comprises at least one salt of hyaluronic acid.

Preferably, said at least one salt of alginic acid is an inorganic salt of alginic acid, even more preferably is sodium alginate.

Preferably said at least one salt of hyaluronic acid is an inorganic salt of hyaluronic acid, even more preferably is sodium hyaluronate.

As mentioned above and as well known to the person of ordinary skill, when the sodium alginate arrives in the stomach and comes into contact with its acid content, it forms a water-insoluble polymer which tends to mix with the food and precipitate within the acidic stomach contents, passing in this way within the gut and making it ineffective its action. In order for the action of the sodium alginate interposed mechanically between the acid contents of the stomach and esophageal walls to be effective, the alginate polymer must be able to place on top of the stomach contents, and not inside it, so as to form a sort of “floating barrier.” Advantageously, thanks to the fact that the nutritional or pharmaceutical composition according to the invention comprises a salt of alginic acid (such as for example sodium alginate) in combination with a salt of hyaluronic acid (such as sodium hyaluronate), it allows a quick and effective positioning of the alginate polymer above the contents of the stomach, in order to form said barrier, as explained in detail below.

From the chemical point of view, the hyaluronic acid is a linear polysaccharide chain, produced by the concatenation of thousands of disaccharide units, connected by bonds β-(1→4) and formed by residues of glucuronic acid and N-acetylglucosamine, in turn linked by ties β-(1→3). Thanks to its particular chemical structure, hyaluronic acid is able to bind to itself many water molecules, reaching a high degree of hydration.

Advantageously, therefore, the hyaluronic acid, derived from the dissociation of sodium hyaluronate in water, forms a gel that tends to float. Surprisingly, according to the invention, the Applicant has found that the composition of sodium alginate and sodium hyaluronate allows the formation of the polymer of alginate and hyaluronic acid gel simultaneously, thus allowing the rapid incorporation of the alginate within the polymer hyaluronic acid gel, before the polymer alginate precipitates within the contents of the stomach acid.

Thus, thanks to the rapid formation of the hyaluronic acid gel, a protective barrier of spongy gel is created that floats above the contents of the stomach acid. Advantageously, said protective barrier of spongy gel, floating above the contents of the stomach acid, adheres on the fundus of the stomach, on the lower esophageal sphincter and on the terminal part of the esophagus thereby forming a protective barrier that mechanically protects the walls of the mucosa by the possible gastroesophageal erosion of acidic stomach contents during cases of reflux.

Advantageously, unlike the compositions known within the prior art that exploit the formation of the gaseous carbon dioxide to facilitate the flotation of the alginate polymer above the acid contents of the stomach and which have high margins of unpredictability, the nutritional or pharmaceutical composition according to the invention ensures a quick and immediate incorporation of the alginate within the polymer hyaluronic acid gel just formed and consequently the creation of the protective barrier of spongy gel which, floating above the contents of the stomach acid, protects the mucosa of the gastro-walls.

Advantageously, the sodium hyaluronate comprised in the composition according to a preferred embodiment of the invention has a molecular weight between 1,000,000 and 2,000,000 Dalton and even more preferably has a molecular weight of 1,500,000 Dalton. The use of sodium hyaluronate having a molecular weight value comprised within the range mentioned above is essential for the rapid formation of the hyaluronic acid gel. In fact, the speed of hydration of hyaluronic acid into contact with water to form the gel, depends on the molecular weight; the molecules having a lower molecular weight do not guarantee an optimal formation of the gel, while the molecules with a higher molecular weight are almost insoluble in water already at low concentrations.

It follows that the use of sodium hyaluronate having a molecular weight comprised within the above range ensures the formation by dissociation of hyaluronic acid having molecular weight optimal for the rapid formation of an internal stomach gel.

In a preferred embodiment, the nutritional or pharmaceutical composition according to the present invention comprises preferably sodium alginate and sodium hyaluronate in a proportion by weight preferably between 5:1 and 200:1, more preferably between 20:1 and 30:1 and even more preferably equal to about 25:1.

The nutritional or pharmaceutical composition according to the invention therefore has the advantage of being able to be used for the treatment or prevention of gastroesophageal reflux disease by operating as the protective barrier that mechanically protects the mucosa of the gastro-walls from the possible erosion by acid in the contents of the stomach.

According to the invention a dietary supplement is also provided.

Note that in this context the term “dietary supplement” means a food product intended to supplement the common diet and which is a concentrated source of nutrients such as vitamins and minerals, or other substances with a nutritional or physiological effect, in particular, but not limited to, amino acids, organic and inorganic salts, essential fatty acids, fiber and various plant-derived extracts, alone or in combination, in unit dose form.

The dietary supplement according to the invention is preferably in the form of free or compressed liquid or powder or capsule and even more preferably is in the form of liquid and includes a nutritional or pharmaceutical composition for the treatment or prevention of gastroesophageal reflux disease comprising at least one salt of alginic acid and at least one salt of hyaluronic acid.

According to a preferred embodiment of the invention said dietary supplement has a pH value between 4.5 and 8.5.

According to a preferred embodiment of the invention said dietary supplement preferably comprises sodium alginate in a concentration percentage by weight which varies between 1% and 3%, even more preferably wherein said sodium alginate is in a concentration percentage by weight equal to 2.29%, and preferably comprises sodium hyaluronate—in particular sodium hyaluronate preferably having molecular weight of 1,500,000 Dalton—in a concentration percentage by weight which varies between 0.020% and 0.200%, wherein even more preferably said sodium hyaluronate is in a concentration percentage by weight equal to 0.090%.

According to a preferred embodiment of the invention the dietary supplement further comprises one or more excipients such as sugar alcohols such as xylitol and other polyols, flavor, water, glyceryl stearate citrate or analogs (as an emulsifier), sucralose, pH adjusters and/or preservatives.

By way of a non-limiting example, a food supplement in the form of liquid according to the invention may comprise: sodium alginate in a concentration percentage by weight of 2.29%; sodium hyaluronate having a molecular weight 1,500,000 Dalton and in percentage concentration by weight of 0.090%; polyol in a concentration percentage by weight of 4.67%; flavor such as aroma pear MK A2945 concentration in weight percentage of 0.08%; water concentration in weight percentage of 92.68%; glyceryl stearate citrate in concentration percentage by weight of 0.18%; and sucralose in a concentration percentage by weight of 0.01%.

The dietary supplement according to the present disclosure can be prepared according to the method described below: providing the at least one salt of alginic acid and the at least one salt of hyaluronic acid; providing one or more excipients; weighing the at least one salt of alginic acid, the at least one salt of hyaluronic acid and said excipients; heating a mixer to 95° C.; placing in said mixer and mixing said at least one salt of alginic acid, said at least one salt of hyaluronic acid and said excipients; cooling the mixer, and the mixture thus obtained, at a temperature comprising between 20° C. and 25° C.

By way of non-limiting example, the dietary supplement of the example above can be prepared according to the method described below: prepare sodium alginate and sodium hyaluronate having a molecular weight 1,500,000 Dalton; prepare the following excipients: polyol, pear aroma MK A2945, water, glyceryl stearate citrate, sucralose; weigh 0.250 g of sodium alginate sodium, 0.010 g of sodium hyaluronic acid with a molecular weight of 1,500,000 Dalton, 0.510 g of polyol, 0.009 g of pear aroma MK A2945, 10.110 g of water, 0.020 g of glyceryl stearate citrate, 0.001 g of sucralose; heat the mixer up to 95° C.; into said mixer, add and mix the water and the glyceryl stearate citrate until completely dissolved; in said mixer add the sodium hyaluronate having a molecular weight 1,500,000 Dalton and let dissolve; in said mixer add the sodium alginate and allow to dissolve, then add the polyol and let dissolve; cool the mixer, and the mixture thus obtained, at a temperature between 35° C. and 40° C. and then add the aroma pear MK A2945; cool the mixer, and the mixture thus obtained, at a temperature between 20° C. and 25° C.

The non-limiting example of a dietary supplement thus obtained has, at the end of the preparation method described above, the characteristics of being a slightly opaque solution, semi-dense and slightly opalescent.

The nutritional or pharmaceutical composition and the dietary supplement as described are subject to numerous changes and modifications within the reach of the person of ordinary skill in the art and fall within the scope of the present invention as defined in the present disclosure.

1-10. (canceled)

11. A nutritional or pharmaceutical composition for the treatment or prevention of gastroesophageal reflux disease comprising at least one salt of alginic acid, characterized in that it further comprises at least one salt of hyaluronic acid.

12. The composition according to claim 11, wherein said at least one salt of alginic acid is an inorganic salt of alginic acid, preferably sodium alginate.

13. The composition according to claim 11, wherein said at least one salt of hyaluronic acid is an inorganic salt of hyaluronic acid, preferably sodium hyaluronate.

14. The composition according to claim 12, wherein said at least one salt of hyaluronic acid is an inorganic salt of hyaluronic acid, preferably sodium hyaluronate.

15. The composition according to claim 14, wherein said inorganic salt of hyaluronic acid is sodium hyaluronate and said sodium hyaluronate has a molecular weight ranging between 1000000 Dalton and 2000000 Dalton, preferably equal to 1500000 Dalton.

16. The composition according to claim 15, wherein said salt of alginic acid and said salt of hyaluronic acid are present in said composition in a ratio by weight comprised between 5:1 and 200:1, preferably comprised between 20:1 and 30:1, even more preferably equal to about 25:1.

17. A food supplement of the liquid or free powder or tablet or capsule type, characterized in that it comprises a nutritional or pharmaceutical composition according claim 16.

18. The food supplement according to claim 17, wherein said salt of alginic acid is in a percentage concentration by weight ranging between 1% and 3%, and preferably equal to 2.29%, and wherein said salt of hyaluronic acid is in a percentage concentration by weight ranging between 0.020% and 0.200%, and preferably equal to 0.090%.

19. The food supplement according to claim 17, wherein said food supplement has a pH value comprised between 4.5 and 8.5.

20. The food supplement according to claim 18, wherein said food supplement has a pH value comprised between 4.5 and 8.5.

21. Use of a composition according to claim 16 for the treatment or prevention of gastroesophageal reflux disease.

22. Use of a food supplement according to claim 20 for the treatment or prevention of gastroesophageal reflux disease.
